(19) United States
(12) Patent Application Publication
(10) Pub. No.: US 2020/0069295 A1
(43) Pub. Date: Mar. 5, 2020

(54) HYDROPHOBIC GAS PERMEABLE FILTER ASSEMBLY FOR MICROFILTRATION OF EXHALED GASES

(71) Applicant: SALTER LABS, Carlsbad, CA (US)

(72) Inventors: Benjamin Walter WANG, Carlsbad, CA (US); Michael Sean SMITH, Oceanside, CA (US); Lorelee Kae GOEHLE, Oceanside, CA (US)

(21) Appl. No.: 16/116,155

(22) Filed: Aug. 29, 2018

(51) Int. Cl. A61B 10/00; B01D 46/00

(52) U.S. Cl. CPC A61B 10/00; A61B 2010/0087; B01D 46/0012

(57) ABSTRACT

A filter assembly for filtering an exhaust gas and preventing contaminates from reaching a medical sampling instrumentation. The filter assembly comprises a hydrophobic filter media component; a first enclosure with an inlet (second) port communicating with an inwardly facing surface carrying a plurality of first channels, a first annular sidewall surrounding the first channels, and the first annular sidewall carrying a first mating feature at a free end thereof; a second enclosure having an outlet (first) port which communicating with an inwardly facing surface carrying a plurality of second channels, and a second mating feature surrounding the second channels. The first and second enclosures matingly engaging with one another so that the first and the second mating features mate with one another and the first and second enclosures define a filter chamber therebetween which captively retains the hydrophobic filter media component within the filter chamber having minimal dead space.

### FIELD OF THE INVENTION

The present invention relates to a custom filter assembly which utilizes a conventional “off-the-shelf” hydrophobic filter media component in which the custom filter assembly is designed to have a very low internal volume or dead space so as to minimize, during use, turbulent flow of a gas flowing through the hydrophobic filter media component.

### BACKGROUND OF THE INVENTION

As is well known in the art, inline filters are provided for microfiltration of exhaled gases used for medical sampling applications. Such filter are designed to prevent moisture and other undesirable particles from flowing/ingressing into medical sampling instrumentation. It is to be appreciated that if undesired liquid and particles ingress into medical instrumentation, such ingress eventually leads to loss of functionality of the medical sampling instrumentation and/or damage to the medical sampling instrumentation.

Long term usage of medical sampling devices, such as an EtCO₂ sampling cannula, necessitates the filtration and separation of moisture as well as other contaminants from the sampled gas, prior to that sampled gas entering into a sampling port of the medical sampling instrumentation. It is to be appreciated that exhaled breath, which mixes with ambient air that is colder than body temperature, will increase the relative humidity of that moisture rich exhaled breath. If the relative humidity rises to 100%, for example, then condensation of the breath typically occurs. Such condensation will be drawn in, via an inlet of a sampling device, e.g., a cannula, and flow towards the hydrophobic filter which is located downstream of the sampling device but upstream of the medical sampling instrumentation, to filter and remove this undesired moisture.

As is well known in the art, moisture and other biohazard contaminants, such as microbes, mucosal secretions, skin cells, hair, particulates, etc., can flow along a sampling line and be delivered to the medical sampling instrumentation together with the collected gas sample. It is to be appreciated that such contaminants can degrade the sensor electronics and/or create potential occlusions within the medical instrument itself thereby adversely affecting the performance and/or accuracy of the medical instrumentation.

While a number of off-the-shelf filter assemblies, equipped with a hydrophobic media, are currently available, they suffer from a number of associated drawbacks. For example, the internal volume of such know filter assemblies, which accommodate the hydrophobic media, are not optimized and thus such known filter assemblies tend to cause undesired mixing and turbulent flow of the exhaled gases, within the filter assembly, which leads to a compromised waveform as well as inaccurate measurements by the medical sampling instrumentation.

It is to be appreciated that a hydrophilic media promotes the transfer of liquids which defeats the purpose of a moisture barrier designed to protect the medical sampling instrumentation. It is noted that hydrophilic media is generally available in greater supply and in different formats (e.g., hollow fiber, membrane, etc.) due to higher demand in the liquid processing industry. Hydrophobic filter media, on the other hand, is not as readily available and this, in turn, makes sourcing off-the-shelf turnkey filter assemblies somewhat more challenging, difficult and expensive.

### SUMMARY OF THE INVENTION

Wherefore, it is an object of the present disclosure is to overcome the above mentioned shortcomings and drawbacks associated with the prior art filter assemblies.

Another object of the present disclosure is to provide a custom filter assembly which utilizes a conventional off-the-shelf hydrophobic filter media component in which the custom filter assembly is designed to have a very low internal volume or dead space and is also designed to reduce turbulent flow through the hydrophobic filter media component while the exhaled gas is filtered by the filter assembly.

A further object of the present disclosure is to captively retain the hydrophobic filter media component, between the inwardly facing surfaces of the first and the second enclosures, and thereby minimize the associated volume or dead space of the internal chamber which is defined by and between the inwardly facing surfaces of the first and the second enclosures.

Still another object of the present disclosure is to minimize the associated expense and labor in connection with manufacturing and assembling the custom filter assembly.

Yet another object of the present disclosure is to sandwich a conventional off-the-shelf hydrophobic filter media component between the pair of inwardly facing surfaces of the first and the second enclosures so as to prevent movement thereof.

A still further object of the present disclosure is to induce generally laminar flow along the channels and through the custom filter assembly so that the exhaust gas is filtered, by the custom filter assembly, on a first in/first out basis with the exhaust gas experiencing minimal turbulence as such gas flows through the filter assembly.

A further object of the disclosure is to provide a custom filter system which is capable of filtering exhaust gases at the rate of about 50 millimeters per minute.

The present invention also relates to a filter assembly for filtering an exhaust gas and preventing a contaminate from reaching a medical sampling instrumentation, the filter assembly comprising: a hydrophobic filter media component; a first enclosure having a first port communicating with an inwardly facing surface carrying a plurality of first channels, a first annular sidewall surrounding the first channels, and the first annular sidewall carrying a mating feature at a free end thereof; a second enclosure having a second port communicating with an inwardly facing surface carrying a plurality of second channels, and a mating second feature surrounding the second channels; and the first and second enclosures matingly engaging with one another so that the first and the second mating features mate with one another and the first and second enclosures define a sealed filter chamber therebetween which captively retains the hydrophobic filter media component within the filter chamber such that the filter assembly has minimal dead space.

The present invention also relates to a method of filtering an exhaust gas and preventing a contaminate from reaching a medical sampling instrumentation, the method comprising: providing a hydrophobic filter media component; providing a first enclosure having a first port communicating with an inwardly facing surface carrying a plurality of first channels, a first annular sidewall surrounding the first channels, and the first annular sidewall carrying a first feature at a free end thereof; providing a second enclosure having a second port communicating with an inwardly facing surface carrying a plurality of second channels, and a second mating feature surrounding the second channels; and matingly engaging the first and second enclosures with one another so that the first and the second mating features mate with one another and the first and second enclosures define a sealed filter chamber therebetween which captively retains the hydrophobic filter media component within the filter chamber such that the filter assembly has minimal dead space

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. The invention will now be described, by way of example, with reference to the accompanying drawings in which:

FIG. 1 is a diagrammatic top, front, left side perspective view of the custom filter assembly, according to the disclosure, shown in its assembled state;

FIG. 2 is a diagrammatic top plan view of the custom filter assembly of FIG. 1;

FIG. 2A is a diagrammatic cross sectional view of the custom filter assembly of FIG. 2 along section line 2A-2A;

FIG. 3 is a diagrammatic front elevational view of the custom filter assembly of FIG. 1;

FIG. 3A is a diagrammatic cross sectional view of the custom filter assembly of FIG. 3 along section line 3A-3A;

FIG. 4 is a diagrammatic left side view of the custom filter assembly of FIG. 1;

FIG. 5 is a diagrammatic exploded view of FIG. 1 showing the components utilized to assemble the custom filter assembly along with a gas sampling device, a gas supply line, a filtered gas line and medical sampling instrumentation diagrammatically shown;

FIG. 6 is a diagrammatic top, front, left side perspective view of the first enclosure;

FIG. 7 is a diagrammatic top plan view of FIG. 6;

FIG. 8 is a diagrammatic front elevational view of FIG. 6;

FIG. 9 is a diagrammatic left side elevational view of the first enclosure of FIG. 6;

FIG. 10 is a diagrammatic bottom plan view of FIG. 6;

FIG. 11 is a diagrammatic top, front, left side perspective view of the first enclosure;

FIG. 12 is a diagrammatic top plan view of FIG. 11;

FIG. 13 is a diagrammatic front elevational view of FIG. 11;

FIG. 14 is a diagrammatic left side view of the first enclosure of FIG. 11; and

FIG. 15 is a diagrammatic bottom plan view of FIG. 11.

It should be understood that the drawings are not necessarily to scale and that the disclosed embodiments are sometimes illustrated diagrammatical and in partial views. In certain instances, details which are not necessary for an understanding of this disclosure or which render other details difficult to perceive may have been omitted. It should be understood, of course, that this disclosure is not limited to the particular embodiments illustrated herein.

### DETAILED DESCRIPTION OF THE PREFERRED EMBODIMENTS

The present invention will be understood by reference to the following detailed description, which should be read in conjunction with the appended drawings. It is to be appreciated that the following detailed description of an embodiment is by way of example only and is not meant to limit, in any way, the scope of the present invention.

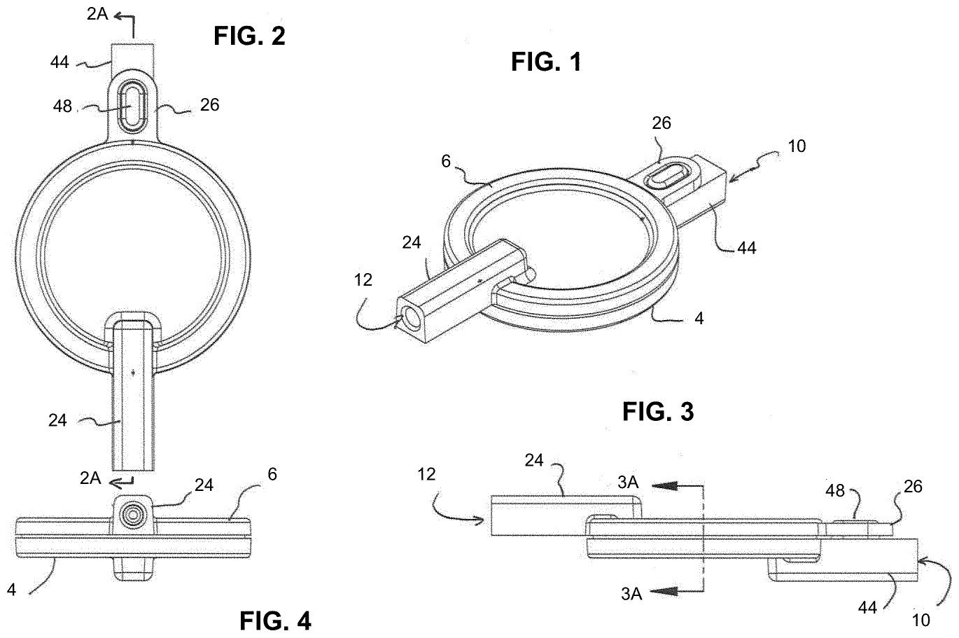

Turning first to FIGS. 1-5, a brief description concerning the various components of the custom filter assembly 2 will now be briefly discussed. As can be seen in this embodiment, the custom filter assembly 2 comprises both a first enclosure 4 and a mating second enclosure 6 which, when joined with one another by ultrasonic welding for example, as discussed below in further detail, captive retain and sandwich a conventional off-the-shelf hydrophobic filter media component 8 therebetween. As diagrammatically shown in FIG. 5, the second enclosure 6 has an inlet (second) port 10 for receiving the exhaust gas from a gas sampling device 11, e.g., a cannula, while the first enclosure 4 has an outlet (first) port 12 for discharging the filtered exhaust gas from the custom filter assembly 2 and supplying the same to a desired medical sampling instrumentation 13, e.g., a capnography monitor. An exhaust gas sampling tubing 15 (only diagrammatically shown) connects an outlet of the gas sampling device 11 to the inlet (second) port 10 while a filtered gas tubing 17 (only diagrammatically shown) connects the outlet (first) port 12 to an inlet of the medical sampling instrumentation 13 for discharging the filtered exhaust gas from the custom filter assembly 2 and supplying the same thereto.

While the second enclosure 6 is described as being the inlet (second) port 10 for receiving the exhaust gas from a gas sampling device 11 and the first enclosure 4 is described as being the outlet (first) port 12 for discharging the filtered exhaust gas from the custom filter assembly 2 and supplying the same to a desired medical sampling instrumentation 13, it is to be appreciated that their rolls may be reversed. That is, the first (outlet) port 12 of the first enclosure 4 may be connected to the gas sampling device 11 for receiving the exhaust gas therefrom while the second (inlet) port 10 of the second enclosure 6 may be connected to medical sampling instrumentation 13 for discharging the filtered exhaust gas from the custom filter assembly 2 and supplying the same thereto, without departing from the spirit and scope of the present invention.

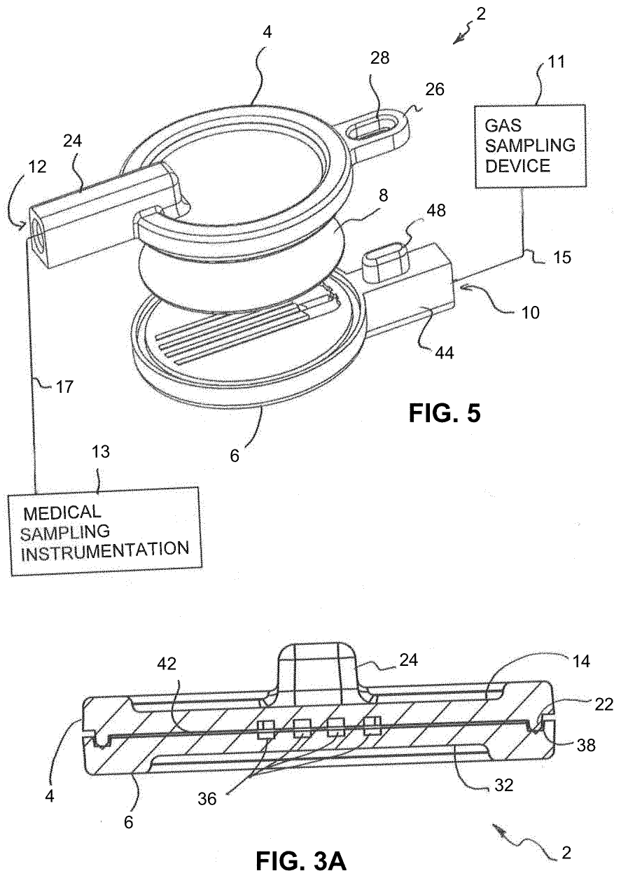

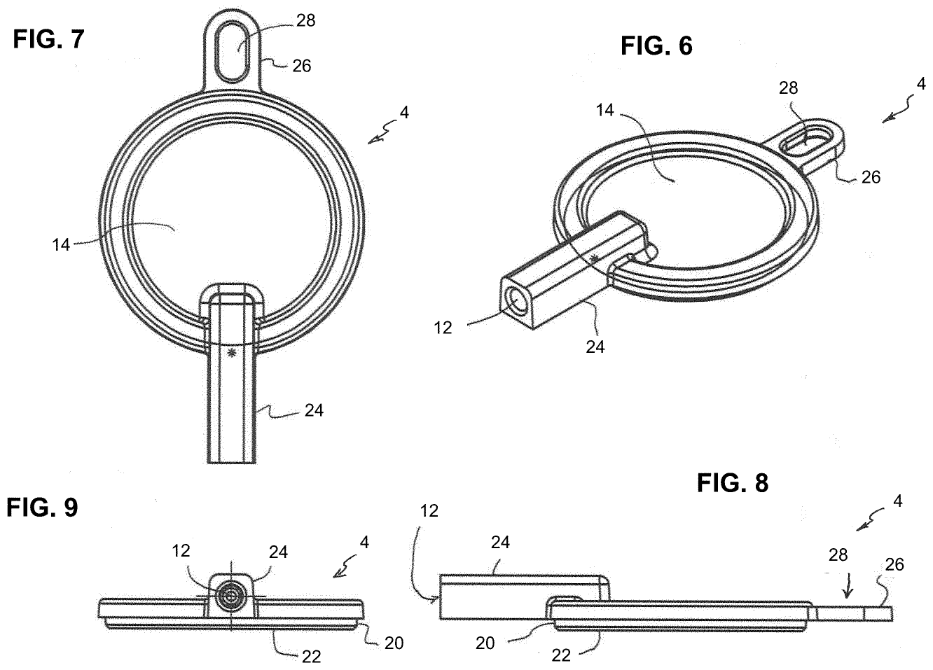

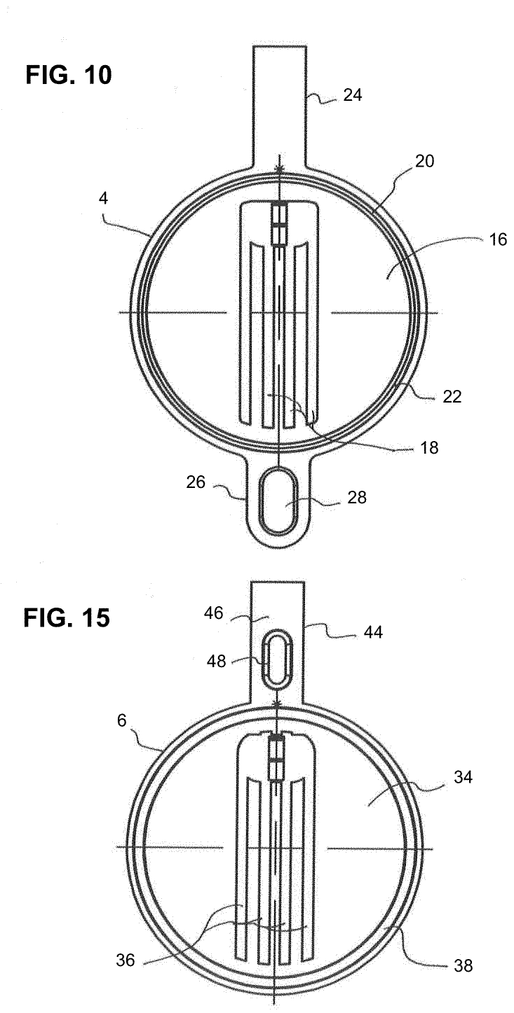

Now turning to FIGS. 6-10 of the drawings, a detail description concerning the first enclosure 4 will now be provided. The first enclosure 4 is generally a low profile component which has a generally flat or planar outwardly facing surface 14 as well as an opposed inwardly facing surface 16 thereof which is also generally flat or planar (see FIG. 10). As best shown in FIGS. 3A, 5 and 10, a plurality of spaced apart first channels 18 are formed in the inwardly facing surface 16 of the first enclosure 4. Each one of the first channels 18 generally extends parallel to one another and parallel to a flow axis defined by the inlet second (inlet) and the outlet ports 10, 12 of the filter assembly 2. Each first channel 18 typically has a width of between 0.060 inches and 0.015 inches, generally about 0.039 inches, and a depth of between 0.050 inches and 0.010 inches, generally about 0.021 inches. Each of the first channels 18 is spaced apart from one or more adjacent first channels 18 by a distance of between 0.060 inches and 0.015 inches, generally about 0.039 inches. The first channels 18 are designed to receive the exhaust gases, once the same passes through and is filtered by the hydrophobic filter media component 8, and redirect such filter exhaust gases along the length of the first channels 18 of the first enclosure 4 toward the outlet (first) port 12 while, at the same time, minimizing turbulence as the exhaust gases flow through the first channels 18 of the first enclosure 4.

An annular sidewall 20 surrounds in the inwardly facing surface 16 of the first enclosure 4 and extends substantially normal thereto. A remote, free end of this annular sidewall 20 carries a tapering tip or an annular tongue 22, the purpose and function of this annular tongue 22 will become apparent from the following description. The annular sidewall 20 is shaped and sized to closely receive and accommodate a perimeter surface of the hydrophobic filter media component 8 on the inwardly facing surface 16 of the first enclosure 4. The annular sidewall 20 typically has a height of between 0.060 inches and 0.015 inches, generally about 0.032 inches.

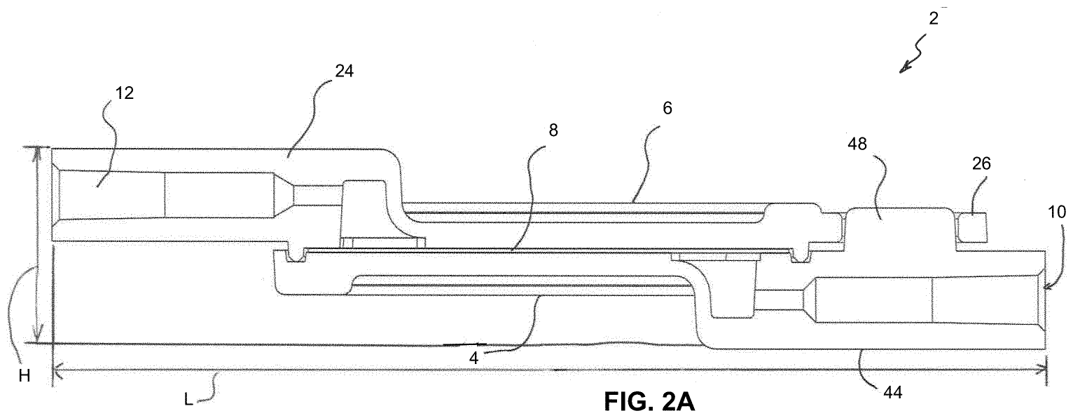

A (first) outlet extension 24 is formed integral with and extends away from a main body of the first enclosure 4 and this outlet extension 24 defines the exhaust gas first (outlet) port 12 of the custom filter assembly 2. As shown in FIG. 2A, the first (outlet) port 12 commences with a first opening, formed in the inwardly facing surface 16 of the first enclosure 4, and the first (outlet) port 12 then reduces in size or diameter and turns or bends, e.g., at a 30 to 90 degree angle, and extends centrally through and along the entire length of the outlet extension 24 and terminates at a second opening which supply the exhaust gases from the first channels 18 of the first enclosure 4 to the first (outlet) port 12. As shown, a diameter of the first (outlet) port 12 eventually transitions to a larger diameter adjacent a free end of the outlet extension 24. The outlet extension 24 typically has a length of between 0.025 inches and 0.090 inches, typically about 0.60 inches.

A second extension 26 extends away from the first enclosure 4 in an opposite direction to the first outlet extension 24. As shown in FIGS. 6-8 and 10, the second extension 26 is typically axially shorter in length and thinner in thickness than the (first) outlet extension 24. The second extension 26 carries a centrally located first (female) interlocking feature 28, e.g., an elongate slot or some other interlocking feature, which facilitates interconnection of the first enclosure 4 with a mating feature 30, e.g., a mating boss for example, carried by the second enclosure 6 to prevent relative rotation between the two enclosures 4, 6 with respect to one another, and a further discussion concerning the same will be provided below.

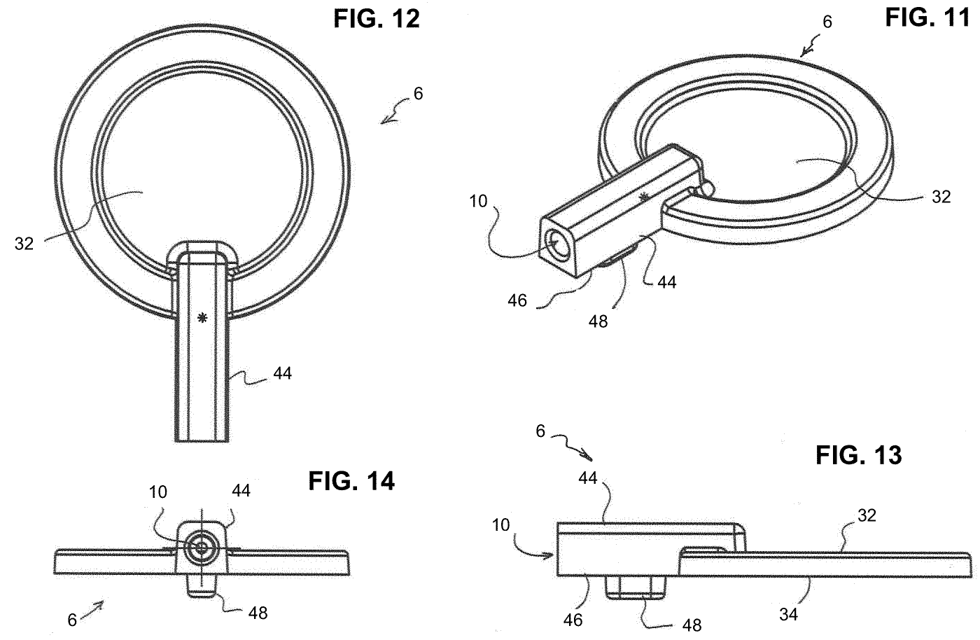

Turning now to FIGS. 11-15 of the drawings, a detail description concerning the second enclosure 6 will now be provided. The second enclosure 6 is also a generally a low profile member which has a generally flat or planar outwardly facing surface 32 and an opposed inwardly facing surface 34 which is also generally flat or planar. As best shown in FIGS. 3A and 15, a plurality of spaced apart second channels 36 are formed in the inwardly facing surface 34 of the second enclosure 6. Each one of the second channels 36 generally extends parallel to one another and parallel to the flow axis defined by the second (inlet) and the outlet (first) ports 10, 12 of the filter assembly 2. Each second channel 36 typically has a width of between 0.060 inches and 0.015 inches, generally about 0.039 inches, and a depth of between 0.050 inches and 0.010 inches, generally about 0.021 inches. Each one of the second channels 36 is spaced apart from one another by a distance of between 0.060 inches and 0.015 inches, generally about 0.039 inches. The second channels 36 are arranged and designed to receive and distribute the exhaust gases supplied by the second (inlet) port 10 along the length of the second channels 18 of the second enclosure 6 while reducing turbulence therein.

A mating annular groove 38 is formed in the inwardly facing surface 34 of the second enclosure 6 and this annular groove 38 extends substantially normal to the inwardly facing surface 34. The mating annular groove 38 is sized, shaped and located to receive and captively retain the annular tongue 22 of the first enclosure 4 during assembly. The annular groove 38 is also sized and shaped so as to be slightly larger in size than a perimeter surface of the hydrophobic filter media component 8 so as to facilitate completely receiving, accommodating and captively retaining the same. The annular groove 38 typically has a depth of between 0.030 inches and 0.005 inches, generally about 0.019 inches. If desired, the sidewalls of the annular groove 38 may taper inwardly somewhat toward one another. When the first and the second enclosures 4, 6 mate with one another, as shown in FIG. 5 for example, the annular tongue 22 is received by and within the mating annular groove 38 so that the first and the second enclosures 4, 6 thereby sandwich and captively retain the hydrophobic filter media component 8 therebetween.

After the first and the second enclosures 4, 6 are assembled with one another as generally shown in FIGS. 1-4, for example, the inwardly facing surfaces 16, 34 of the first and the second enclosures 4, 6 together with the annular sidewall 20, the mating annular tongue 22 and the annular groove 40 define an internal filter chamber 42 which has a minimal volume, e.g., a total internal volume of the filter chamber 42 is typically between 0.114 mL (minimum) and 0.165 mL liters (maximum), typically about 0.139 mL and thus has very little dead space.

As shown in FIGS. 3 and 4 for example, the second (inlet) port 10 is axially offset with respect to the outlet (first) port 12 by distance slightly larger than the thickness of the filter chamber 42.

Since the lateral regions of both the first and second enclosures 4, 6 are not provided with any first or second channels 18, 36, substantially none of the supplied exhaust gases will flow or be filtered by such lateral regions of the hydrophobic filter media component 8. That is, all of the filtering of the exhaust gases occurs primarily through the hydrophobic filter media component 8 which is located between and separates the first channels 18 from the second channels 36.

An inlet extension 44 extends away from a main body of the second enclosure 6 and this inlet extension 44 defines the exhaust gas second (inlet) port 10 for receiving exhaust gases to be filtered by the filter assembly 2. The second (inlet) port 10 typically has a constant diameter along the length thereof which then transitions into a reduce diameter before the second (inlet) port 10 eventually turns or bends and then terminates as an opening formed in the inwardly facing surface 34 of the second enclosure 6. As shown, after the bend or turn, the size or the diameter of the inlet (second) port 10 again increases in size.

An outwardly facing surface 46 of the inlet extension 44 carries a second (male) interlocking feature 48, e.g., an oval shaped boss or some other interlocking feature, which is sized and shaped to mate closely with and be received by the centrally located first (female) interlocking feature 28, e.g., the elongate slot of the first enclosure 4, to thereby couple and interconnect the first and second enclosures 4, 6 with one another. The mating engagement between the mating male and female or interlocking features 28, 48 prevents rotation of the first enclosure 4 relative to the second enclosure 6.

Once the first and the second enclosures 4, 6 are connected with one another, as generally shown in FIG. 1, then the entire perimeter of the annular tongue 22 and the annular groove 40 are ultrasonically welded to one another thereby to form the sealed filtered chamber 42 with the hydrophobic filter media component 8 being captively retained therein. In addition, the mating male and female or interlocking features 28, 48 are also ultrasonically welded to one another to secure further the engagement between the first enclosure 4 with the second enclosure 6 and also prevent relative rotation or separation of the first enclosure 4 and the second enclosure 6 from one another.

After the mating male and female or interlocking features 28, 48 are welded together, the hydrophobic filter media component 8 occupies substantially all of the spaced defined within the filtered chamber 42 except for the first and the second channels 18, 36, thereby minimizing the unoccupied volume or the dead space contained within the filtered chamber 42. That is, a first surface of the hydrophobic filter media component 8 generally directly engages with the inwardly facing surface 16 of the first enclosure 4, or is possibly spaced a very small distance therefrom, e.g., less than 0.010 of an inch and more preferably less than 0.005 of an inch, while a second surface of the hydrophobic filter media component 8 generally directly engages with the inwardly facing surface 34 of the second enclosure 6, or is possibly spaced a very small distance therefrom, e.g., less than 0.010 of an inch and more preferably less than 0.005 of an inch.

The first channels 18 together define a total volume of between 0.0632 mL (minimum) and 0.103 mL (maximum), typically about 0.0843 mL, while the second channels 36 together also define a total volume of between 0.0632 mL (minimum) and 0.103 mL (maximum), typically about 0.0843 mL and the hydrophobic filter media component 8 separates first channels 18 from the second channels 36. The thickness of the custom filter assembly 2, measured from the outwardly facing surface 14 of the first enclosure 4 to the outwardly facing surface 32 of the second enclosure 6, is generally between 0.665 inches and 0.250 inches, typically about 0.372 inches, and is thus low profile.

The conventional off-the-shelf hydrophobic filter media component 8 typically has a diameter of less than 1.0 inch, generally less than 0.996 inches, a thickness typically between 155 μm (minimum) and 185 μm (maximum), generally about 170 μm and a porosity of about 0.2 microns. The hydrophobic filter media component 8 is designed to filter the supplied exhaust gases and remove moisture and other contaminants, such as microbes, mucosal secretions, skin cells, hair, particulates, etc., therefrom as the exhaust gases pass through the hydrophobic filter media component 8 of the filter assembly 2 and thereby prevent such moisture and contaminants from flowing toward and into the medical sampling instrumentation 13.

While both the first and the second enclosures 4, 6 are shown as being circular in shape and the annular sidewall of the first enclosure 4 is shown as being substantially cylindrical in shape, it is to be appreciated that the first and the second enclosures 4, 6 and the annular sidewall of the first enclosure 4 can have a variety of other different shapes and sizes without departing from the spirit and scope of the present invention. The most important aspect is that the first and the second enclosures 4, 6 together define a sealed filtering chamber 42 therebetween which defines a minimal dead space therein.

While the above disclosure indicates that the filter assembly 2 has four parallel channels 18, 36, it is to be appreciated that the overall number, size, location, shape, etc., of each one of the channels 18, 36 can varied from application to application without departing from the spirit and scope of the present invention. The important aspect is that the channels 18, 36 are designed to reduce the overall size of the dead space within the filter chamber 42 as well as minimize turbulence of the exhaust gases as such gases flow through and are filtered by the off-the-shelf hydrophobic filter media component 8 within the filter chamber 42 of the filter assembly 2.

It is to be appreciated that the annular tongue 22 and the annular groove 40 may, instead of being welded, possibly be glued, fused, or otherwise permanently affixed or connected to one another in a conventional manner to form the sealed filtered chamber 42 with the hydrophobic filter media component 8 being captively retained therein, without departing from the spirt and scope of the present invention.

Preferably the hydrophobic filter media component 8 is relatively thin and is disc shaped so as to be closely and captively received by and between the inwardly facing surfaces of the first and second enclosures 4, 6.

The first and second enclosures 4, 6 are preferably each injection molded from a plastic material, such as acrylonitrile butadiene styrene (ABS), acrylic, polycarbonate, etc. Due to the low profile of the filter assembly 2, the overall axial length L of the filter assembly 2, from an end face of the outlet (first) port 12 to an end face of the second (inlet) port 10, is at least three times overall height H of the filter assembly 2—see FIG. 2A. More preferably, the overall axial length L of the filter assembly 2 is at least four times the overall height H of the filter assembly 2. Most preferably, the overall axial length L of the filter assembly 2 is at least five times, and approaching seven times, the overall height H of the filter assembly 2.

While a single embodiment of the present invention is described in detail, it is apparent that various modifications and alterations of that embodiment will occur to and be readily apparent to those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having,” and variations thereof herein, is meant to encompass the items listed thereafter and equivalents thereof as well as additional items while only the terms “consisting of” and “consisting only of” are to be construed in a limitative sense.

The foregoing description of the embodiment of the present disclosure is presented for the purposes of illustration and description. It is not intended to be exhaustive or to limit the present disclosure to the precise form disclosed. Many modifications and variations are possible in light of this disclosure. It is intended that the scope of the present disclosure be limited not by this detailed description, but rather by the claims appended hereto.

Wherefore, I/We claim:

1. A filter assembly for filtering an exhaust gas and preventing a contaminate from reaching a medical sampling instrumentation, the filter assembly comprising:
a hydrophobic filter media component;
a first enclosure having a first port communicating with an inwardly facing surface carrying a plurality of first channels, a first annular sidewall surrounding the first channels, and the first annular sidewall carrying a first feature at a free end thereof;
a second enclosure having a second port communicating with an inwardly facing surface carrying a plurality of second channels, and a mating second feature surrounding the second channels; and
the first and second enclosures matingly engaging with one another so that the first and the second mating features mate with one another and the first and second enclosures define a sealed filter chamber therebetween which captively retains the hydrophobic filter media component within the filter chamber such that the filter assembly has minimal dead space.

2. The filter assembly according to claim 1, wherein both the first and the second enclosures are both low profile members which have a generally planar outwardly facing surface and a generally planar inwardly facing surface.

3. The filter assembly according to claim 1, wherein each one of the first channels extend parallel to one another and each one of the second channels extend parallel to one another.

4. The filter assembly according to claim 1, wherein each of the first and the second channels has a width of between 0.060 inches and 0.015 inches and a depth of between 0.050 inches and 0.010 inches, and each one of the first channels are spaced apart from one another by a distance of between 0.060 inches and 0.015 inches.

5. The filter assembly according to claim 1, wherein first mating feature is an annular tongue located at a free end of the annular sidewall while the second mating feature is a mating annular groove.

6. The filter assembly according to claim 1, wherein the first enclosure has a first extension which defines the first port, and the first port extends through an entire length of the first extension and then bends toward and passes through an opening formed in the inwardly facing surface of the first enclosure for supplying the exhaust gases directly to the plurality of first channels of the first enclosure.

7. The filter assembly according to claim 6, wherein the first enclosure has a second extension which extends away from the first enclosure, in an opposite direction to the first extension, and the second extension carries a first interlocking feature for preventing rotation of the first and the second enclosures relative to one another.

8. The filter assembly according to claim 1, wherein the filter chamber has an internal volume, for the exhaust gas, which is between 0.114 mL and 0.165 mL.

9. The filter assembly according to claim 1, wherein an outlet extension extends away from the second enclosure and the outlet extension defines the second port for discharging the filtered exhaust gases from the filter assembly, and the second port comprises an opening formed in the inwardly facing surface of the second enclosure and then bends and extends through and along the outlet extension for supplying the filtered exhaust gases from the second channels to the medical sampling instrumentation.

10. The filter assembly according to claim 7, wherein an outwardly facing surface of the outlet extension carries a second interlocking feature which is sized and shaped to mate with a first interlocking feature of the second extension of the first enclosure and thereby couple and connect the first and the second enclosures with one another and prevent relative rotation with respect to one another.

11. The filter assembly according to claim 1, wherein the hydrophobic filter media component is a member which has a thickness of between about 155 μm and about 185 μm and a porosity of about 0.2 microns.

12. The filter assembly according to claim 1, wherein both the first and the second enclosures are generally circular shaped components.

13. The filter assembly according to claim 1, wherein the first enclosure has four first channels and the second enclosure has four second channels and the hydrophobic filter media component separates the first channels from the second channels.

14. The filter assembly according to claim 1, wherein a thickness of the filter chamber axially offsets and spaces the first port from the second port.

15. The filter assembly according to claim 1, wherein an overall axial length of the filter assembly is at least three times an overall height of the filter assembly.

16. The filter assembly according to claim 1, wherein a first surface of the hydrophobic filter media component directly engages with the inwardly facing surface of the first enclosure while an opposed second surface of the hydrophobic filter media component directly engages with the inwardly facing surface of the second enclosure.

17. The filter assembly according to claim 1, wherein a first surface of the hydrophobic filter media component is spaced a small distance from the inwardly facing surface of the first enclosure while an opposed second surface of the hydrophobic filter media component is spaced a small distance from the inwardly facing surface of the second enclosure.

18. The filter assembly according to claim 1, wherein of the custom filter assembly has a thickness, measured from an outwardly facing surface of the first enclosure to the outwardly facing surface of the second enclosure, of between 0.665 inches and 0.250 inches.

19. A filter assembly for filtering an exhaust gas and preventing a contaminate from reaching a medical sampling instrumentation, the filter assembly comprising:
a hydrophobic filter media component;
a first enclosure having a first port communicating with an inwardly facing surface carrying a plurality of first channels;
a second enclosure having a second port communicating with an inwardly facing surface carrying a plurality of second channels; and
the first and second enclosures matingly engaging, with one another with the hydrophobic filter media component located therebetween, to define a sealed filter chamber which captively retains the hydrophobic filter media component while defining an internal filter chamber having a dead space of between 0.114 mL and 0.165 mL so as to minimize turbulent flow of the exhaust gas though the filter assembly.

20. A method of filtering an exhaust gas and preventing a contaminate from reaching a medical sampling instrumentation, the method comprising:
providing a hydrophobic filter media component;
providing a first enclosure having a first port communicating with an inwardly facing surface carrying a plurality of first channels, a first annular sidewall surrounding the first channels, and the first annular sidewall carrying a first feature at a free end thereof;
providing a second enclosure having a second port communicating with an inwardly facing surface carrying a plurality of second channels, and a mating second feature surrounding the second channels; and
matingly engaging the first and second enclosures with one another so that the first and the second mating features mate with one another and the first and second enclosures define a sealed filter chamber therebetween which captively retains the hydrophobic filter media component within the filter chamber such that the filter assembly has minimal dead space.
